(19) United States
(12) Patent Application Publication
(10) Pub. No.: US 2009/0090383 A1
(43) Pub. Date: Apr. 9, 2009

(54) METHOD AND APPARATUS FOR CLEANING AN INTEGRATING SPHERE

(76) Inventor: Alan Ingleson, Newbury (GB)

Correspondence Address: PATTERSON & SHERIDAN L.L.P. NJ Office, 3040 Oak Post Road, Suite 1500, Houston, TX, 77056-6582, US

(21) Appl. No.: 12/245,178

(22) Filed: Oct. 3, 2008

Related U.S. Application Data
(60) Provisional application No. 60/978,582, filed on Oct. 9, 2007.

(51) Int. Cl. B08B 7/00; G01J 3/00

(52) U.S. Cl. 134/1.1; 356/319

(57) ABSTRACT

In one embodiment, the present invention is a method and apparatus for cleaning an integrating sphere, such as an integrating sphere used in an integrating sphere spectrophotometer. One embodiment of a spectrophotometer includes an integrating sphere having a reflective interior surface, a primary light source configured to illuminate the interior surface when enabled, and a secondary light source configured to emit ionizing radiation onto the interior surface.

### CROSS REFERENCE TO RELATED APPLICATIONS

This application claims the benefit of U.S. Provisional Patent Application Ser. No. 60/978,582, filed Oct. 9, 2007, which is herein incorporated by reference in its entirety.

### BACKGROUND OF THE DISCLOSURE

The white interior surfaces of integrating spheres that are typically used in spectrophotometers are prone to yellowing due to bacterial action on the binder substrate of the surfaces. This yellowing results in the incursion of geometric errors and the magnification of other errors when the integrating sphere is in use. These errors may be so bad that the spectrophotometer cannot be corrected by software to behave like an integrating-sphere spectrophotometer that has a new white sphere.

### SUMMARY OF THE INVENTION

In one embodiment, the present invention is a method and apparatus for cleaning an integrating sphere, such as an integrating sphere used in an integrating sphere spectrophotometer. One embodiment of a spectrophotometer includes an integrating sphere having a reflective interior surface, a primary light source configured to illuminate the interior surface when enabled, and a secondary light source configured to emit ionizing radiation onto the interior surface.

### BRIEF DESCRIPTION OF THE DRAWINGS

The teachings of the present invention can be readily understood by considering the following detailed description in conjunction with the accompanying drawings, in which:

FIG. 1 is a schematic diagram illustrating one embodiment of an integrating sphere spectrophotometer according to the present invention;

FIG. 2 is a flow diagram illustrating one embodiment of a method for cleaning the reflective interior surface of an integrating sphere, according to the present invention;

FIG. 3 is a flow diagram illustrating one embodiment of a method for monitoring the reflective properties of a specular port of an integrating sphere spectrophotometer, according to the present invention;

FIG. 4 is a flow diagram illustrating one embodiment of a method for calibrating the spectrophotometer illustrated in FIG. 1, according to one embodiment of the present invention; and

FIG. 5 is a high level block diagram of the integrating sphere cleaning method that is implemented using a general purpose computing device.

To facilitate understanding, identical reference numerals have been used, where possible, to designate identical elements that are common to the figures.

### DETAILED DESCRIPTION

In one embodiment, the invention is a method and apparatus for cleaning an integrating sphere, such as an integrating sphere used in a fielded integrating-sphere spectrophotometer. Embodiments of the invention utilize a source of ionizing radiation, such as an ultraviolet light, to clean and whiten the matte optical surface of the integrating sphere, which was originally white but has yellowed with age.

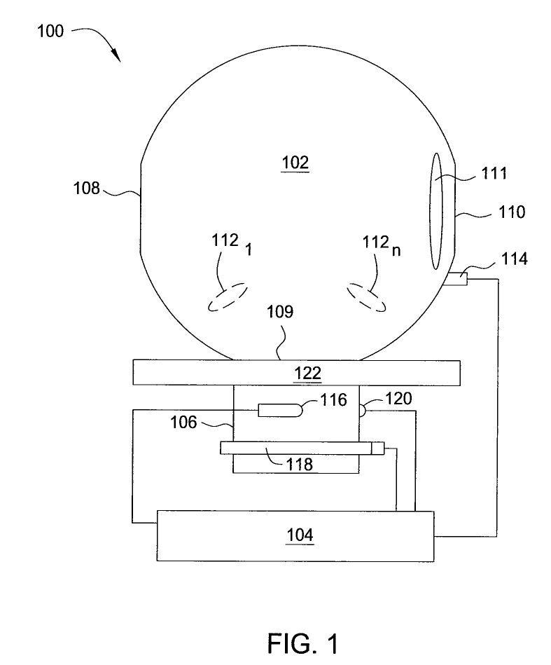

FIG. 1 is a schematic diagram illustrating one embodiment of an integrating sphere spectrophotometer 100 according to the present invention. As illustrated, the spectrophotometer 100 comprises an integrating sphere 102 that is coupled to a controller (computer) 104 and to an illumination housing 106.

The integrating sphere 102 comprises an interior wall having a high-reflectance, matte optical surface. In one embodiment, the integrating sphere 102 comprises a sample input port 108 formed in a side of the sphere and a sample exit port 110 formed in another side of the sphere opposite the sample input port 108. A specular port 111 is formed in the integrating sphere 102 and positioned near the sample exit port 110. An illuminant port 109 is also formed in the integrating sphere 102 and is positioned in a plane that is substantially perpendicular to a plane in which the sample input port 108 and the sample exit port 110 are positioned. In a further embodiment, the integrating sphere 102 additionally comprises one or more baffles 112₁-112ₙ (hereinafter collectively referred to as “baffles 112” disposed inside the sphere. In addition, the integrating sphere 102 comprises a sphere sensor 114 positioned near the sample exit port 110. In one embodiment, the sphere sensor comprises a dispersive element, such as a grating. As illustrated, the sphere sensor 114 is coupled to the controller 104.

The illumination housing 106 is coupled to the integrating sphere 102 via the illumination port 109. In one embodiment, the illumination housing 106 comprises a primary light source 116 positioned near the integrating sphere 102 and a secondary light source 118 positioned on an opposite side of the primary light source 116 from the integrating sphere 102 (i.e., such that the primary light source 116 is positioned between the integrating sphere 102 and the secondary light source 118). Both the primary light source 116 and the secondary light source 118 are coupled to the controller 104. In one embodiment, the primary light 116 source is a pulsed xenon flash or a light-emitting diode (LED). The secondary light source 118 is an ionizing radiation source, and in one embodiment comprises an ultraviolet (UV) light, an inert gas (e.g., neon) light, or a mercury vapor lamp. In one embodiment, the secondary light source 118 comprises multiple atomic (spectral) lines. For example, if the secondary light source 118 is a mercury vapor lamp, mercury vapor gives rise to substantial power at wavelengths of 254 nm, 365 nm, 435.8 nm, and 546.1 nm; if the secondary light source comprises neon, it will comprise atomic lines at even higher wavelengths. In addition, the illumination housing 106 comprises a source sensor 120 coupled to the illumination housing 106 and to the controller 104.

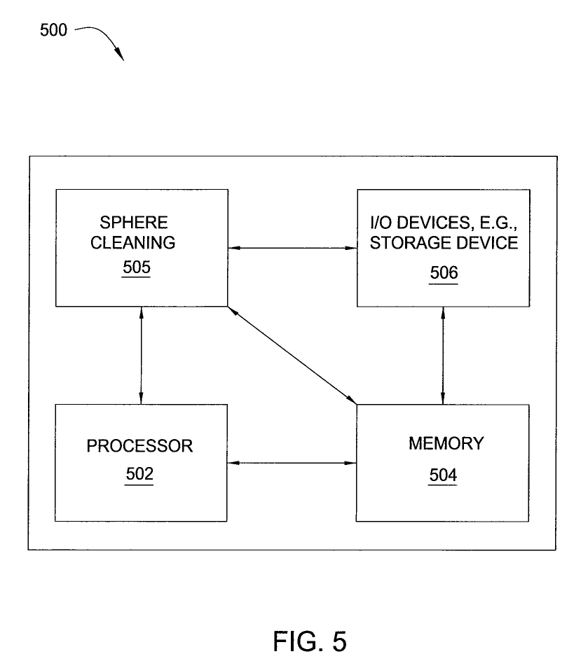

In one embodiment, the controller 104 is a general purpose computer, such as, for example, the general purpose computer described in further detail with respect to FIG. 5.

In one embodiment, an optional filter wheel 122 is coupled to the illumination port 109 of the integrating sphere 102, such that the filter wheel 122 is positioned between the integrating sphere 102 and the illumination housing 106. In one embodiment, the filter wheel 122 is a UV filter wheel.

In operation, the primary light source 116 illuminates the integrating sphere 102 through the illumination housing 106 and the illumination port 109. The illumination provided by the primary light source 116 may be guided in the integrating sphere 102 by the baffles 112, if the baffles 112 are included in the integrating sphere 102. The illumination provided by the primary light source 116 is diffused in the integrating sphere 102, and the diffused illumination is collected by the sphere sensor 114, through the sample exit port 110. The diffused light collected by the sensor is then used (e.g., by the controller 104 or by a multi-pixel detector, not shown) to analyze the reflectance information from a sample (not shown) positioned near the sample input port 108. If the filter wheel 122 is included in the spectrophotometer 100, the filter wheel 122 is used to adjust the short-wavelength light to mimic daylight (e.g., Commission Internationale de l'Éclairage (CIE) D65 illumination). This adjustment is needed to measure fluorescent samples.

The dispersive element of the sphere sensor disperses the collected illumination into component wavelengths. A mutli-pixel detector (for example integrated into the controller 104) then measures, at each wavelength, the illumination that exits the integrating sphere 102 via the sample exit port 110. By comparing this measurement with a measurement of illumination from a reference beam (not shown), the spectrophotometer 100 can infer the reflectance of the sample.

As discussed above, the reflective interior surface of the integrating sphere 102, which is white when new, may yellow over time due to bacterial action. However, the configuration of the spectrophotometer 100 allows the interior surface of the integrating sphere 102 to be whitened or cleaned by a non-contact bactericidal action involving irradiation of the surface.

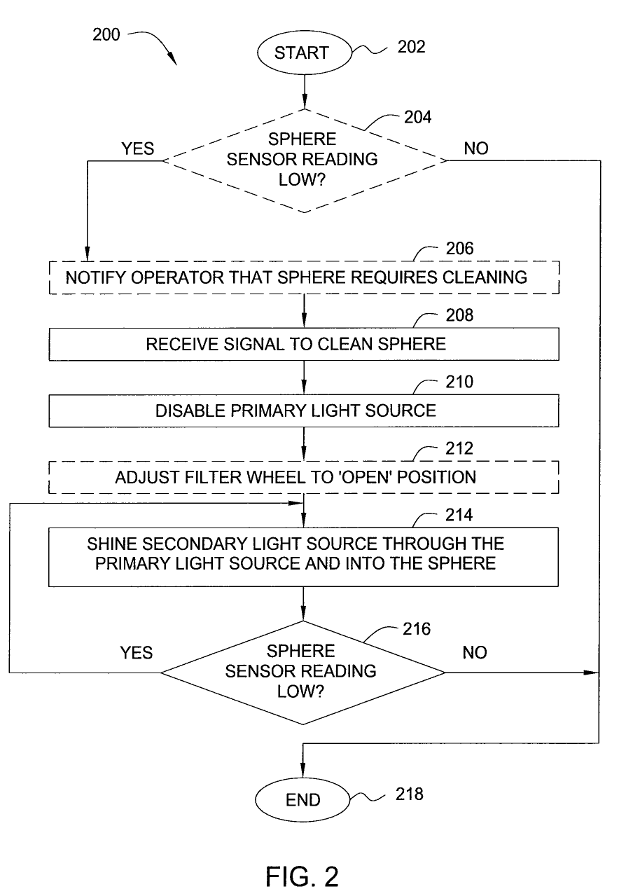

FIG. 2 is a flow diagram illustrating one embodiment of a method 200 for cleaning the reflective interior surface of an integrating sphere, according to the present invention. The method 200 may be implemented, for example, by the controller 104 of FIG. 1 in order to clean the interior surface of the integrating sphere 102. As such, discussion of the method 200 will refer to the components of the spectrophotometer 100 illustrated in FIG. 1. It is to be appreciated, however, that that operation of the method 200 is not limited to use with the spectrophotometer configuration illustrated in FIG. 1.

The method 200 is initialized at step 202 and proceeds to optional step 204 (illustrated in phantom), where the controller 104 determines whether the sphere sensor 114 reads low (e.g., showing a reduction of approximately ten percent compared to when the integrating sphere 102 was new). The sphere sensor 114 measures the radiation in the integrating sphere 102; thus, a low reading by the sphere sensor 115 could indicate absorption of UV light from the integrating sphere 102.

If the controller 104 concludes in step 204 that the sphere sensor 114 reads low, then the integrating sphere 102 is assumed to require cleaning. In this case, the method 200 proceeds to optional step 206 (illustrated in phantom) and notifies an operator (e.g., a human operator) that the integrating sphere 102 requires cleaning.

Alternatively, if the controller 104 concludes in step 204 that the sphere sensor 114 does not read low, the method 200 terminates in step 218. In an alternative embodiment, rather than terminate the method 200, the controller 104 may continue to monitor the readings from the sphere sensor 114 until a low reading is again detected.

It is noted that steps 204-206 are designated as optional. In an embodiment where the need to clean the integrating sphere 102 is automatically detected, steps 204-206 are used to facilitate the automatic detection and notification. However, in embodiments where the need to clean the integrating sphere 102 is not automatically detected, cleaning may be initiated on-demand, for example in response to a request from a human operator. In such embodiments, the method 200 may skip steps 204-206 and proceed directly to step 208 from initialization at step 202.

In step 208, the controller 104 receives a signal (e.g., from a human operator) to clean the integrating sphere 102. In step 210, the controller 104 disables (i.e., turns off) the primary light source 116, such that no illumination is emitted by the primary light source 116.

In optional step 212 (illustrated in phantom), the controller 104 adjusts the filter wheel 122 to the “open” position. Step 212 is optional because, in some embodiments, the spectrophotometer 100 will not include the filter wheel 122.

Once the primary light source 116 is disabled and the filter wheel is adjusted (if necessary), the method 200 proceeds to step 214, where the controller 104 enables (i.e., turns on) the secondary light source 118, such that the secondary light source 118 shines through the primary light source 116 and into the integrating sphere 102. Continued exposure of the integrating sphere's interior surface to the ionizing radiation emitted from the secondary light source 118 will gradually whiten the integrating sphere's interior surface (i.e., by killing the bacteria that cause the yellowing). In one embodiment, surfaces other than the interior surface of the integrating sphere 102 are shielded from the ionizing radiation emitted by the secondary light source 118.

In step 216, the controller 104 again determines whether the sphere sensor 114 still reads low (e.g., below the predefined threshold). If the controller 104 concludes in step 216 that the sphere sensor 114 still reads low, then the integrating sphere 102 is assumed to require further cleaning. In this case, the method 200 returns to step 214, and the controller 104 continues to shine the secondary light source 118 into the integrating sphere 102.

Alternatively, if the controller 104 concludes in step 216 that the sphere sensor 114 no longer reads low, the cleaning process is assumed to have finished. In this case, the method 200 terminates in step 218. In an alternative embodiment, rather than terminate the method 200, the controller 104 may continue to monitor the readings from the sphere sensor 114 until a low reading is again detected. In another embodiment, if the cleaning process is determined not to yield satisfactory results after a predetermined period of time (i.e., after a predetermined period of exposure of the integrating sphere's interior surface to the ionizing radiation), then the method 200 is terminated, and the integrating sphere 102 is assumed to require replacement.

The method 200 therefore provides a non-destructive method of whitening the interior surface of the integrating sphere 102, using bactericidal action. The interior surface is whitened by continued exposure to the ionizing radiation emitted by the secondary light source 118, which kills the bacteria responsible for the yellowing. Moreover, because the method 200 requires substantially no physical contact with the interior surface of the integrating sphere 102 (as might be required, for example, if one were to wash or wipe the surface), the risk of damage to the interior surface of the integrating sphere 102 is substantially minimized. The fidelity of the spectrophotometer 100 relative to a master instrument can thus be renewed, with any residual difference being correctable by software. The integrating sphere 102 may be re-radiated periodically using the secondary light source 118 in order to preserve the whiteness of the interior surface.

Moreover, in embodiments where a mercury vapor lamp is used as the secondary light source 118, toxic ozone emissions can be substantially reduced. The strong 254 nm atomic line of mercury vapor is in the middle of a useful wavelength range for germicidal light, but is also outside of the spectral region (e.g., less than approximately 242 nm) that produces ozone emissions. Furthermore, the primary ozone-producing radiation in a mercury lamp is due to power at less than 200 nm, which is readily filtered out by commercially available ozone-free mercury pen lamps.

In addition to enabling cleaning of the integrating sphere 102, the ionizing radiation provided by the secondary light source 118 can be used to monitor the reflective properties of the specular port 111. For instance, a specular port that does not function correctly may require cleaning or replacement.

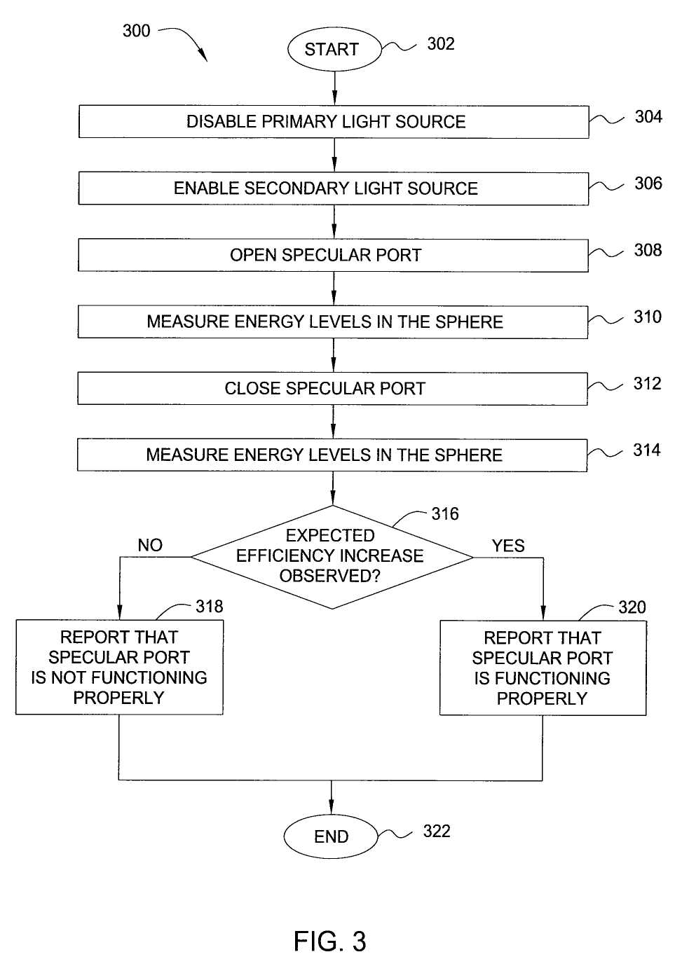

FIG. 3 is a flow diagram illustrating one embodiment of a method 300 for monitoring the reflective properties of a specular port of an integrating sphere spectrophotometer, according to the present invention. The method 300 may be implemented, for example, by the controller 104 of FIG. 1 in order to monitor the properties of the specular port 111. As such, discussion of the method 300 will refer to the components of the spectrophotometer 100 illustrated in FIG. 1. It is to be appreciated, however, that that operation of the method 300 is not limited to use with the spectrophotometer configuration illustrated in FIG. 1.

The method 300 is initialized at step 302 and proceeds to step 304, where the controller 104 disables (i.e., turns off) the primary light source 116. The method 300 then proceeds to step 306, where the controller 104 enables (i.e., turns on) the secondary light source 118, so that the secondary light source 118 emits ionizing radiation into the integrating sphere 102.

In step 308, the controller 104 opens the specular port 111 of the integrating sphere 102. The method 300 then proceeds to step 310, where the controller 104 measures the energy levels in the integrating sphere 102 with the specular port 111 open. In one embodiment, the controller 104 uses the sphere sensor 114 and the source sensor 120 to measure the energy levels.

In step 312, the controller 104 closes the specular port 111. The controller 104 then measures the energy levels in the integrating sphere again in step 314, this time with the specular port 111 closed. In one embodiment, the controller 104 uses the sphere sensor 114 and the source sensor 120 to measure the energy levels.

In step 316, the controller determines whether the increase in efficiency of the integrating sphere 102 (i.e., as measured by the sphere sensor 114 and the source sensor 120) matches an expected increase in efficiency for a highly reflective specular port. If the controller 104 concludes in step 316 that the expected efficiency increase has not been observed, the method 300 proceeds to step 318, and the controller 104 reports (e.g., to an operator) that the specular port 111 is not functioning properly.

Alternatively, if the controller 104 concludes in step 316 that the expected efficiency increase has been observed, the method 300 proceeds to step 320, and the controller 104 reports (e.g., to an operator) that the specular port 111 is functioning properly.

Once a report on the functioning of the specular port 111 has been generated (i.e., in accordance with step 318 or 320), the method 300 terminates in step 322.

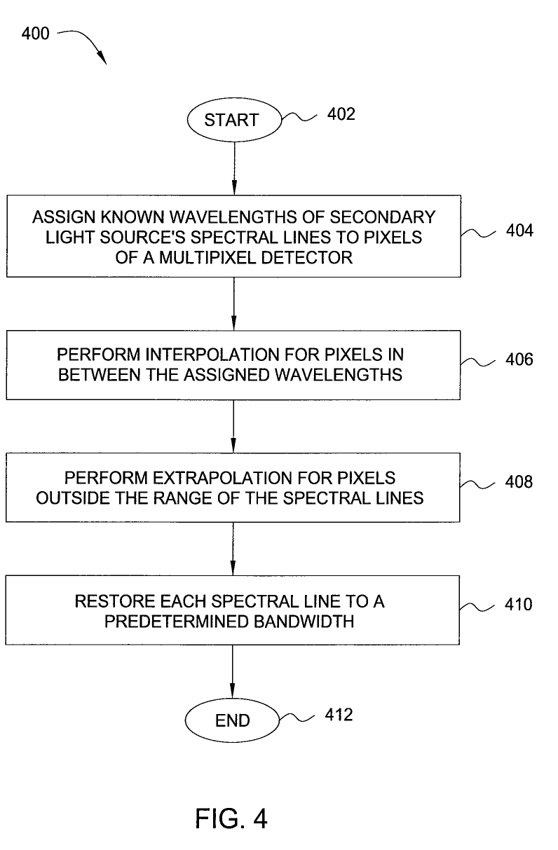

FIG. 4 is a flow diagram illustrating one embodiment of a method 400 for calibrating the spectrophotometer 100 illustrated in FIG. 1, according to one embodiment of the present invention. In one embodiment, calibration in accordance with the method 400 is intended for use as a fielded instrument update where a more careful factory calibration with external light sources has already been performed. The method 400 may be implemented, for example, by the controller 104 illustrated in FIG. 1.

The method 400 is initialized at step 402 and proceeds to step 404, where the controller 104 assigns the known wavelengths of the secondary light source's spectral lines to pixels of a multi-pixel detector (not shown). In step 406, the controller 104 performs interpolation for the pixels that are in between the wavelengths assigned in step 404. Similarly, in step 408, the controller 104 performs extrapolation for the pixels that are outside the range of the secondary light source's spectral lines. Thus, steps 404-408 collectively serve to perform wavelength correction by compensating for wavelength errors in the fielded spectrophotometer 100.

In step 410, the controller 104 restores each of the secondary light source's spectral lines to a predetermined bandwidth. In one embodiment, signal processing is used to perform this restoration. In a further embodiment, standard methods (such as, for example, deconvolution) are used in signal processing to correct for stray light and for other linear spectral distortion, such as bandwidth correction. Thus, step 410 serves to perform bandwidth correction.

The method 400 then terminates in step 412.

FIG. 5 is a high level block diagram of the integrating sphere cleaning method that is implemented using a general purpose computing device 500. In one embodiment, a general purpose computing device 500 comprises a processor 502, a memory 504, a sphere cleaning module 505 and various input/output (I/O) devices 506 such as a display, a keyboard, a mouse, a modem, a network connection and the like. In one embodiment, at least one I/O device is a storage device (e.g., a disk drive, an optical disk drive, a floppy disk drive). It should be understood that the sphere cleaning module 605 can be implemented as a physical device or subsystem that is coupled to a processor through a communication channel.

Alternatively, the sphere cleaning module 505 can be represented by one or more software applications (or even a combination of software and hardware, e.g., using Application Specific Integrated Circuits (ASIC)), where the software is loaded from a storage medium (e.g., I/O devices 506) and operated by the processor 502 in the memory 504 of the general purpose computing device 500. Additionally, the software may run in a distributed or partitioned fashion on two or more computing devices similar to the general purpose computing device 500. Thus, in one embodiment, the sphere cleaning module 505 for cleaning the integrating sphere of a spectrophotometer described herein with reference to the preceding Figures can be stored on a computer readable medium or carrier (e.g., RAM, magnetic or optical drive or diskette, and the like).

It should be noted that although not explicitly specified, one or more steps of the methods described herein may include a storing, displaying and/or outputting step as required for a particular application. In other words, any data, records, fields, and/or intermediate results discussed in the methods can be stored, displayed, and/or outputted to another device as required for a particular application. Furthermore, steps or blocks in the accompanying Figures that recite a determining operation or involve a decision, do not necessarily require that both branches of the determining operation be practiced. In other words, one of the branches of the determining operation can be deemed as an optional step.

Although various embodiments which incorporate the teachings of the present invention have been shown and described in detail herein, those skilled in the art can readily devise many other varied embodiments that still incorporate these teachings.

What is claimed is:

1. A spectrophotometer, comprising:
an integrating sphere having a reflective interior surface;
a primary light source configured to illuminate the interior surface when enabled; and
a secondary light source configured to emit ionizing radiation onto the interior surface.

2. The spectrophotometer of claim 1, wherein the primary light source is a pulsed xenon flash or a light-emitting diode.

3. The spectrophotometer of claim 1, wherein the secondary light source is one of: an ultraviolet light, an inert gas light, and a mercury vapor lamp.

4. The spectrophotometer of claim 1, wherein the secondary light source comprises a plurality of atomic lines.

5. The spectrophotometer of claim 1, wherein the primary light source is positioned between the integrating sphere and the secondary light source.

6. The spectrophotometer of claim 1, wherein the ionizing radiation has a wavelength in a range for germicidal light.

7. A method for cleaning an integrating sphere of an integrating sphere spectrophotometer, comprising:
disabling a primary light source used for illuminating an interior surface of the integrating sphere; and
exposing the interior surface to ionizing radiation.

8. The method of claim 7, wherein the exposing comprises:
shining a secondary light source into the integrating sphere, where the secondary light source emits the ionizing radiation.

9. The method of claim 8, wherein the secondary light source is shined through the primary light source, the primary light source being positioned between the secondary light source and the integrating sphere.

10. The method of claim 7, wherein the secondary light source is one of: an ultraviolet light, an inert gas light, and a mercury vapor lamp.

11. The method of claim 7, wherein the secondary light source comprises a plurality of atomic lines.

12. The method of claim 7, wherein the ionizing radiation has a wavelength in a range for germicidal light.

13. The method of claim 7, further comprising:
determining that the integrating sphere requires cleaning; and
notifying an operator that the integrating sphere requires cleaning.

14. The method of claim 13, wherein the determining comprises:
measuring radiation in the integrating sphere; and
determining that the integrating sphere requires cleaning if the radiation measured in the integrating sphere indicates a reduction of approximately ten percent as compared to when the integrating sphere was new.

15. The method of claim 14, further comprising:
continuing to expose the interior surface to the ionizing radiation until the radiation in the integrating sphere reads below the predefined threshold.

16. The method of claim 7, further comprising:
adjusting a filter wheel positioned between the integrating sphere and a source of the ionizing radiation to an “open” position prior to exposing the interior surface to the ionizing radiation.

17. The method of claim 7, further comprising:
periodically repeating the disabling and the exposing to preserve a whiteness of the interior surface.

18. The method of claim 7, wherein the integrating sphere is cleaned in response to a signal requesting that the integrating sphere be cleaned.

19. The method of claim 7, further comprising:
shielding surfaces other than the interior surface of the integrating sphere from the ionizing radiation.

20. A computer readable storage medium containing an executable program for cleaning an integrating sphere of an integrating sphere spectrophotometer, where the program performs the steps of:
disabling a primary light source used for illuminating an interior surface of the integrating sphere; and
exposing the interior surface to ionizing radiation.
